(19) United States
(12) Patent Application Publication
(10) Pub. No.: US 2008/0147022 A1
(43) Pub. Date: Jun. 19, 2008

(54) Skin care delivery device having a releasable backing

(75) Inventors: Walter G. Bauer, Neenah, WI (US); Craig M. Thoms, Appleton, WI (US); Kroy D. Johnson, Menasha, WI (US); Ed John Foley, Greenville, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/640,432

(22) Filed: Dec. 15, 2006

(51) Int. Cl. A61K 9/70; A61K 35/00; A61P 17/00; A61Q 19/00

(52) U.S. Cl. 604/304; 514/789

(57) ABSTRACT

A skin care delivery device is disclosed. The skin care delivery device includes a substrate containing a skin care composition. The skin care composition can comprise, for instance, any suitable lotion or other similar material. In accordance with the present disclosure, a backing is releasably engaged to one surface of the substrate. The backing facilitates folding and unfolding of the delivery device. Once applied to a user's skin, the backing can be peeled away and discarded.

### BACKGROUND

People rely on health, hygiene, and wellness products as part of their everyday lives. For example, some people use formulations to moisturize or otherwise care for their skin. Many individuals apply creams or lotions to their hands, face, or other parts of their bodies. By applying formulations in this way, dry skin or other problems may be addressed. In many applications, the skin care products are sold as a cream or lotion. The products are dispensed from containers onto the hands and then applied to the desired areas on the body. Unfortunately, the above process typically leaves the lotion or cream coated on the hands. Further, in some embodiments, the hands can actually contaminate the lotion or cream prior to application. Furthermore, some formulations may contain ingredients that evaporate or volatilize from the skin, perhaps degrading the effectiveness of the formulation.

One possible approach to dealing with some of the aforementioned issues is to not apply the formulation directly with the hands. Instead, the formulation can be associated with a substrate, with the substrate then being applied to the skin so that some portion of the formulation contacts the skin.

The above approach, however, may raise other issues to address. A substrate that is pretreated with a formulation should be capable of being shipped by a manufacturer or retailer so that the formulation doesn't spread all over the interior of any container in which the substrate is shipped. Otherwise, the user of the substrate may have the formulation all over his or her hands when removing the substrate from the container and any formulation remaining in the container is wasted. In fact, in one current commercial application, a dry substrate is first folded and placed in a package. After being placed into the package, the substrate is then wetted with a lotion. In this embodiment, in order to ensure that the substrate is completely wetted, the substrate is typically oversaturated. Consequently, excess lotion tends to drip off the substrate during use.

In view of the above, a need currently exists for a skin care delivery device that is easy to handle and manipulate. A need also exists for a disposable skin care delivery device that can be placed onto a desired location by a user with minimal exposure to the hands of the user.

### SUMMARY

In general, the present disclosure is directed to a disposable skin care delivery device and to a method of applying the device. In accordance with the present disclosure, a releasable backing is attached to one surface of a substrate containing a skin care composition. The backing allows a user to handle the substrate with minimal exposure to the skin care composition until the skin care delivery device is properly positioned. For instance, once the user deploys the substrate at a desired body location so that the skin care composition contacts the skin, the user can then remove the backing. The backing also provides numerous other benefits and advantages as will be described in further detail below.

In one embodiment, for instance, the present disclosure is directed to a disposable skin care delivery device that comprises a substrate having a first surface and a second and opposite surface. A skin care composition is contained in the substrate and is present at least at the first surface. In accordance with the present disclosure, a backing is releasably engaged to the second surface of the substrate. The backing is configured to be removed from the substrate during application of the substrate to a user's skin. In one embodiment, for instance, the backing may comprise a polymer film such as a monoextruded film. The polymer film can be made from any suitable polymer, such as any suitable thermoplastic polymer. Particular examples of polymers that may be used to form the polymer film include polyolefins, polyesters, polyvinyls and laminates thereof.

The disposable skin care delivery device can have any suitable shape depending upon the area of the body that is to be treated. In one embodiment, for instance, the disposable skin care delivery device may be in the form of a mask having a shape configured to cover at least a portion of a person's face. The skin care delivery device, for instance, may include first and second eye openings and an opening designed to surround the mouth of a user. In addition, the skin care delivery device may include a nose portion. The nose portion, for instance, may comprise any suitable configuration that allows the nose to fit comfortably under the skin care delivery device. For instance, the nose portion may comprise a slit that allows the substrate to fit around a person's nose.

When the disposable skin care delivery device includes an opening designed to surround a person's mouth, in one embodiment, the opening is formed not only into the substrate, but also in the backing. Eye openings can also be present in the backing that correspond with openings in the substrate.

The disposable skin care delivery device can be packaged in numerous different configurations for delivery to the user. For instance, the disposable skin care delivery device can be at least partially folded upon itself and placed in a sealed package. The package can be made, for instance, from one or more polymer films. In one embodiment, the backing can include at least one tab portion, such as a first tab portion and a second tab portion. Each of the tab portions may be folded over onto the first surface of the substrate. The tab portions can be used to grasp the skin care delivery device and place the skin care delivery device in the appropriate location without having to contact the skin care composition. The tab portions can also be used to unfold the skin care delivery device, especially when the first surface of the substrate is folded upon itself for packaging.

The substrate can generally comprise any suitable material capable of holding and delivering the skin care composition. The substrate may comprise, for instance, a nonwoven web. The nonwoven web, for instance, may contain pulp fibers mixed with synthetic fibers. For instance, in one embodiment, the nonwoven web may comprise a coform web.

Other examples of nonwoven webs that may be used in accordance with the present disclosure include hydroentangled webs, bonded carded webs, air-formed webs, meltblown webs, spunbond webs, and laminates thereof.

The components used to form the skin care composition can depend upon the particular application and the desired result. In one embodiment, for instance, the skin care composition may comprise at least a humectant and a moisturizer. Various other ingredients including medicinal agents can also be included in the composition.

The present disclosure is also directed to a method for treating the skin of a user. The method includes the steps of removing a folded disposable skin care delivery device as described above from a sealed package. After being removed, the skin care delivery device is unfolded using, for instance, the tab portions as described above. Once unfolded, the skin care delivery device is positioned in contact with a user's skin by placing the first surface of the substrate against the skin. After the skin care delivery device is contacted with the skin, the backing is then removed.

Other features and aspects of the present disclosure are discussed in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the following figures:

FIG. 1 is a plan view of one embodiment of a skin care delivery device made in accordance with the present disclosure;

FIGS. 2 through FIGS. 4 illustrate one particular folding configuration of the skin care delivery device shown in FIG. 1 in order to package the device;

FIG. 5 is a plan view of the folded skin care delivery device illustrated in FIG. 4 shown in a sealed package;

FIG. 6 is a perspective view of another embodiment of a skin care delivery device made in accordance with the present disclosure;

FIG. 7 is a perspective view of the skin care delivery device illustrated in FIG. 1 showing the separate layers; and

FIG. 8 is a plan view of another embodiment of a skin care delivery product made in accordance with the present disclosure.

Repeat use of reference characters in the present specification and drawings is intended to represent same or analogous features or elements of the disclosure.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a skin care delivery device for delivering a skin care composition to a user's skin. The skin care delivery device can be used in any suitable location on the body. For instance, in one embodiment, the skin care delivery device may be in the form of a mask used to cover at least a portion of a user's face.

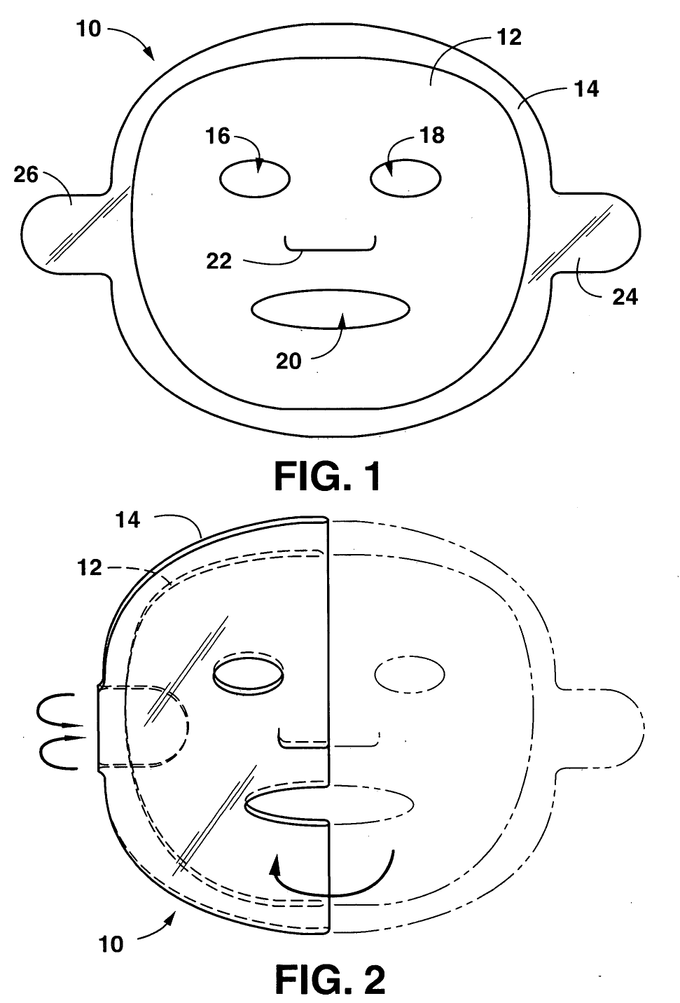

Referring to FIG. 1 and FIG. 7, for instance, one embodiment of a disposable skin care delivery device 10 made in accordance with the present disclosure is shown. In general, the skin care delivery device 10 includes a substrate 12 releasably attached to a backing 14. The substrate 12 contains a skin care composition configured to contact the skin of a user. In accordance with the present disclosure, once the user deploys the substrate 12 at a desired body location so that the skin care composition contacts the skin, the user can then remove the backing 14.

As will be described in greater detail below, depending upon the viscosity, lotion saturation level, and other characteristics of the skin care composition, the substrate 12 and the backing 14 can be folded during manufacture so that the backing associated with the substrate is oriented outward and available for handling by a user. In this way, the skin care delivery device 10 can be more easily shipped without the composition spreading into the interior of any package in which the device is contained. Furthermore, a user can grasp the backing 14 in order to manipulate the skin care delivery device. Once the substrate 12 is deployed at a desired location on a user's body, the backing 14 can be removed. For instance, in one embodiment, the substrate 12 may be breathable. By removing the backing 14, air and/or water vapor may pass through the substrate 12 during use.

As described above, the skin care delivery device of the present disclosure can be used in numerous and various applications. In general, the skin care delivery device can be placed at any suitable location on the body for any suitable therapeutic or medicinal purpose. In the embodiment illustrated in FIG. 1, the skin care delivery device 10 is generally in the form of a mask. For instance, as shown, the substrate 12 includes a pair of spaced apart eye openings 16 and 18. The substrate 12 further includes an opening 20 designed to encircle the mouth of a user. If desired, the substrate 12 can further include a nose portion 22. The nose portion 22 can have any suitable configuration or shape so as to allow the mask to elevate over the nose of a user and/or allow a user to breathe through his or her nose. For instance, as shown in FIG. 1, in one embodiment, the nose portion 22 comprises a slit. In other embodiments, however, the nose portion 22 may comprise a molded section that fits around a user's nose.

The openings 16, 18, 20 and the nose portion 22 can extend only through the substrate 12 or may also extend through the backing 14. In one embodiment, for instance, the mouth opening 20 extends through the entire skin care delivery device 10. In this manner, a user can breathe through his or her mouth during positioning of the skin care delivery device 10 on the user's face.

The substrate 12 as shown in FIG. 1 can be made from any suitable material capable of retaining a skin care composition. For example, the substrate can include a nonwoven fabric, woven fabric, knit fabric, wet-strength paper, or combinations thereof. Materials and processes suitable for forming such substrate are well known to those skilled in the art.

For instance, some examples of nonwoven fabrics that may used in the present disclosure include, but are not limited to, spunbonded webs (apertured or non-apertured), meltblown webs, bonded carded webs, air-formed webs, coform webs, hydraulically entangled webs, and the like. In addition, nonwoven fabrics can contain synthetic fibers (e.g., polyethylenes, polypropylenes, polyvinyl chlorides, polyvinylidene chlorides, polystyrenes, polyesters, polyamides, polyimides, etc.); cellulosic fibers (softwood pulp, hardwood pulp, thermomechanical pulp, etc.); or combinations thereof.

If desired, the nonwoven fabric may also be bonded using techniques well known in the art to improve the durability, strength, hand, aesthetics, texture, and/or other properties of the fabric. For instance, the nonwoven fabric can be thermally (e.g., pattern bonded), ultrasonically, adhesively and/or mechanically (e.g., through-air dried) bonded. For instance, various pattern bonding techniques are described in U.S. Pat. Nos. 3,855,046 to Hansen; 5,620,779 to Levy, et al.; 5,962,112 to Haynes, et al.; 6,093,665 to Sayovitz, et al.; U.S. Design Pat. No. 428,267 to Romano, et al.; and U.S. Design Pat. No. 390,708 to Brown, which are incorporated herein in their entirety by reference thereto for all purposes.

The nonwoven fabric can be bonded by continuous seams or patterns. As additional examples, the nonwoven fabric can be bonded along the periphery of the sheet or simply across the width or cross-direction (CD) of the web adjacent the edges. Other bond techniques, such as a combination of thermal bonding and latex impregnation, may also be used. Alternatively and/or additionally, a resin, latex or adhesive may be applied to the nonwoven fabric by, for example, spraying or printing, and dried to provide the desired bonding. Still other suitable bonding techniques may be described in U.S. Pat. Nos. 5,284,703 to Everhart, et al., 6,103,061 to Anderson, et al., and 6,197,404 to Varona, which are incorporated herein in its entirety by reference thereto for all purposes.

In one embodiment of the present disclosure, the substrate is formed from a nonwoven web containing a mixture of pulp fibers and synthetic fibers. Such substrates can be, for instance, hydroentangled webs or coform webs. Hydroentangling processes and hydroentangled composite webs containing various combinations of different fibers are known in the art. A typical hydroentangling process utilizes high pressure jet streams of water to entangle fibers and/or filaments to form a highly entangled consolidated fibrous structure, e.g., a nonwoven fabric. Hydroentangled nonwoven fabrics of staple length fibers and continuous filaments are disclosed, for example, in U.S. Pat. Nos. 3,494,821 to Evans and 4,144,370 to Bouolton, which are incorporated herein in their entirety by reference thereto for all purposes. Hydroentangled composite nonwoven fabrics of a continuous filament nonwoven web and a pulp layer are disclosed, for example, in U.S. Pat. Nos. 5,284,703 to Everhart, et al. and 6,315,864 to Anderson, et al., which are incorporated herein in their entirety by reference thereto for all purposes.

The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Some examples of such coform materials are disclosed in U.S. Pat. Nos. 4,100,324 to Anderson, et al.; 5,284,703 to Everhart, et al.; and 5,350,624 to Georger, et al.; which are incorporated herein in their entirety by reference thereto for all purposes.

Webs produced by the coform process are generally referred to as coform materials. More particularly, one process for producing coform nonwoven webs involves extruding a molten polymeric material through a die head into fine streams and attenuating the streams by converging flows of high velocity, heated gas (usually air) supplied from nozzles to break the polymer streams into discontinuous microfibers of small diameter. The die head, for instance, can include at least one straight row of extrusion apertures. In general, the microfibers may have an average fiber diameter of up to about 10 microns. The average diameter of the microfibers can be generally greater than about 1 micron, such as from about 2 microns to about 5 microns. While the microfibers are predominantly discontinuous, they generally have a length exceeding that normally associated with staple fibers.

In order to combine the molten polymer fibers with another material, such as pulp fibers, a primary gas stream is merged with a secondary gas stream containing the individualized wood pulp fibers. Thus, the pulp fibers become integrated with the polymer fibers in a single step. The wood pulp fibers can have a length of from about 0.5 millimeters to about 10 millimeters. The integrated air stream is then directed onto a forming surface to air form the nonwoven fabric. The nonwoven fabric, if desired, may be passed into the nip of a pair of vacuum rolls in order to further integrate the two different materials.

When containing cellulosic materials such as pulp fibers, a coform material may contain the cellulosic material in an amount from about 10% by weight to about 80% by weight, such as from about 30% by weight to about 70% by weight. For example, in one embodiment, a coform material may be produced containing pulp fibers in an amount from about 40% by weight to about 60% by weight.

In another embodiment, the substrate is formed from a spunbonded web containing monocomponent and/or multicomponent fibers. Multicomponent fibers are fibers that have been formed from at least two polymer components. Such fibers are usually extruded from separate extruders but spun together to form one fiber. The polymers of the respective components are usually different from each other although multicomponent fibers may include separate components of similar or identical polymeric materials. The individual components are typically arranged in substantially constantly positioned distinct zones across the cross-section of the fiber and extend substantially along the entire length of the fiber. The configuration of such fibers may be, for example, a side-by-side arrangement, a pie arrangement, or any other arrangement. Multicomponent fibers and methods of making the same are taught in U.S. Pat. Nos. 5,108,820 to Kaneko, et al., 4,795,668 to Kruege, et al., 5,162,074 to Hills, 5,277,976 to Hogle, et al., 5,336,552 to Strack, et al., 5,466,410 to Hills, 5,069,970 to Largman, et al., 5,057,368 to Largman, et al., 5,382,400 to Pike, et al., and 5,989,004 to Cook, which are incorporated herein in their entirety by reference thereto for all purposes.

When utilized, multicomponent fibers can also be splittable. In fabricating multicomponent fibers that are splittable, the individual segments that collectively form the unitary multicomponent fiber are contiguous along the longitudinal direction of the multicomponent fiber in a manner such that one or more segments form part of the outer surface of the unitary multicomponent fiber. In other words, one or more segments are exposed along the outer perimeter of the multicomponent fiber. For example, splittable multicomponent fibers and methods for making such fibers are described in U.S. Pat. Nos. 5,935,883 to Pike and 6,200,669 to Marmon, et al., which are incorporated herein in their entirety by reference thereto for all purposes.

In addition, the substrate can also be formed from a material that is imparted with texture one or more surfaces. For instances, in some embodiments, the substrate can be formed from a dual-textured spunbond or meltblown material, such as described in U.S. Pat. Nos. 4,659,609 to Lamers, et al. and 4,833,003 to Win, et al., which are incorporated herein in their entirety by reference thereto for all purposes.

The basis weight of the substrate 12 can vary depending upon the particular application, the particular body part being treated, the ingredients contained in the skin care composition, and various other factors. In general, for instance, the basis weight of the substrate may be from about 20 gsm to about 200 gsm. For instance, in some embodiments, the basis weight may be between about 35 gsm to about 100 gsm.

In accordance with the present disclosure, the substrate 12 can contain any suitable skin care composition. The skin care composition can be contained in the substrate for any suitable purpose that may provide some benefit to the skin. For instance, the skin care composition can be used to moisturize the skin, to provide therapeutic relief to the user, to treat wounds, to treat burns, or to treat any other skin ailments.

In one embodiment, for instance, the skin care composition contains at least one moisturizer and at least one humectant. The moisturizer and/or humectant may be present with one or more surfactants.

In other embodiments, various other beneficial agents may be included in the skin care composition. The classes of ingredients and their corresponding benefits include, without limitation, anti-acne actives (a drug product used to reduce the number of acne blemishes, acne pimples, blackheads, and whiteheads), antifoaming agents (reduce the tendency of foaming during processing), antimicrobial actives, antifungal actives, antiseptic actives, antioxidants (product integrity), cosmetic astringents (include a tightening or tingling sensation on skin), drug astringents (a drug product which checks oozing, discharge, or bleeding when applied to skin or mucous membrane and works by coagulating protein), biological additives (enhance the performance or consumer appeal of the product), colorants (impart color to the product), deodorants (reduce or eliminate unpleasant odor and protect against the formation of malodor on body surfaces), emollients (help to maintain the soft, smooth, and pliable appearance of the skin by their ability to remain on the skin surface or in the stratum corneum to act as lubricant, to reduce flaking, and to improve the skin's appearance), external analgesics (a topically applied drug that has a topical analgesic, anesthetic, or antipruritic effect by depressing cutaneous sensory receptors, or that has a topical counterirritant effect by stimulating cutaneous sensory receptors), film formers (to hold active ingredients on the skin by producing a continuous film on the skin upon drying), fragrances (consumer appeal), opacifiers (reduce the clarity or transparent appearance of the product), skin conditioning agents, skin exfoliating agents (ingredients that increase the rate of skin cell turnover: alpha hydroxy acids and beta hydroxyacids), skin protectants (a drug product which protects injured or exposed skin or mucous membrane surface from harmful or annoying stimuli), solvents (liquids employed to dissolve components found useful in the cosmetics or drugs), sunscreens (ingredient that absorbs at least 85% of the light in the UV range at wavelengths from 290 to 320 nonometers, but transmit UV light at wavelengths longer than 320 nanometers), and surfactants (as cleansing agents, emulsifying agents, solubilizing agents, and suspending agents).

In addition to these classes of ingredients, small amounts (from about 0.01 to about 20%) of oil soluble/dispersible or lipophilic materials can be easily emulsified into the formulation using anionic, cationic, nonionic and/or zwitterionic surfactants. Lipophilic materials without limitation include, silicones/organomodified silicones (protection, tissue water resistance, lubricity, tissue softness; oils (minerals, vegetable, and animal); fatty esters and the like. Powders to enhance lubricity, oil absorption, provide skin protection, astringency, opacity, etc. and microencapsulated ingredients can also be dispersed into the formulation.

In one embodiment, the skin care composition may comprise the following formulation which is particularly well adapted to being contacted with the face of a user.

| | | | Weight % | % Activity | Final Weight | |
|---|---|---|---|---|---|---|
| SUPPLIER(S) | TRADE NAME | CHEMICAL NAME | in Formula | of Ingredient | % in Formulation | Function |
| | Water | Water | 75.110 | 100.00 | 75.110 | Carrier |
| Cognis | Emulgade CM | Cetearyl Isononanoate | 10.000 | 15.00 | 1.500 | Emollient |
| | | Ceteareth-20 | | 7.50 | 0.750 | Surfactant |
| | | Cetearyl alchohol | | 7.50 | 0.750 | Surfactant |
| | | Glyceryl stearate | | 2.50 | 0.250 | Emulsifier |
| | | Glycerin | | 2.50 | 0.250 | Humectant |
| | | Ceteareth-12 | | 0.50 | 0.050 | Surfactant |
| | | Cetyl palmitate | | 0.50 | 0.050 | Occlusive |
| | | Water | | 64.00 | 6.400 | Carrier |
| | Glycerin | Glycerin | 5.000 | 100.00 | 5.000 | Humectant |
| Cognis | Elestab FL-15 | Butylene Glycol | 3.000 | 42.7000 | 1.281 | Moisturizer |
| | | Glycerin | | 42.7000 | 1.281 | Humectant |
| | | Chlorphenesin | | 7.1000 | 0.213 | Preservative |
| | | Methylparaben | | 7.5000 | 0.225 | Preservative |
| | Betafin BP-20 | Betaine | 2.000 | 100.00 | 2.000 | Humectant |
| | 1,3 Butylene Glycol | Butylene Glycol | 1.000 | 100.00 | 1.000 | Moisturizer |
| Active Organics | Actiphyte of Avocado | Butylene Glycol | 0.200 | 39.50 | 0.079 | Moisturizer |
| | BG50P | Water | | 39.50 | 0.079 | Carrier |
| | | Phenoxyethanol | | 0.7260 | 0.001 | Preservative |
| | | Methylparaben | | 0.1550 | 0.000 | Preservative |
| | | Ethylparaben | | 0.0390 | 0.000 | Preservative |
| | | Propylparaben | | 0.0200 | 0.000 | Preservative |
| | | Butylparaben | | 0.0400 | 0.000 | Preservative |
| | | Isobutylparaben | | 0.0200 | 0.000 | Preservative |
| | | Persea Gratissima (Avocado) Fruit | | 20.00 | 0.040 | Avocado |
| | | Extract | | | | |
| Dow Coming | DC 193 Fluid | PEG-12 dimethicone | 0.500 | 100.00 | 0.500 | Emoliient |
| Rhodia | Jaguar C-13S | Guar hydroxypropyltrimonium | 0.100 | 100.00 | 0.100 | Conditioning |
| | | chloride | | | | agent |
| Firmenich | Mandrin Ginger Flowers | Fragrance | 0.600 | 100.00 | 0.600 | Fragrance |
| | 178470B | | | | | |
| Active Organics | Actiphyte of Jojoba | Butylene Glycol | 0.200 | 39.50 | 0.079 | Moisturizer |
| | Meal BG50P | Water | | 39.50 | 0.079 | Carrier |
| | | Phenoxyethanol | | 0.7260 | 0.001 | Preservative |
| | | Methylparaben | | 0.1550 | 0.000 | Preservative |
| | | Ethylparaben | | 0.0390 | 0.000 | Preservative |
| | | Propylparaben | | 0.0200 | 0.000 | Preservative |
| | | Butylparaben | | 0.0400 | 0.000 | Preservative |
| | | Isobutylparaben | | 0.0200 | 0.000 | Preservative |
| | | Simmondsia Chinensis (Jojoba) Seed | | 20.00 | 0.040 | Jojoba |
| | | Extract | | | | |
| | | Disodium EDTA | 0.050 | 100.00 | 0.050 | Chelating |
| | | | | | | agent |
| Seppic | Simulgel NS | Hydroxyethyl acrylate/Sodium | 1.560 | 37.50 | 0.585 | |
| | | acrylydimethyltaurate copolymer | | | | |
| | | Sqalane | | 25.50 | 0.398 | |
| | | Polysorbate 60 | | 5.50 | 0.086 | |
| | | Water | | 31.50 | 0.491 | |
| | Potassium hydroxide | Potassium hydroxide | 0.180 | 10.000 | 0.018 | |
| | 10% solution | Water | | 90.000 | 0.162 | |
| PentaPharm | 80% Phytaluronate/20% | Ceratonia Siliqua Gum | 0.100 | | 0.000 | |
| | Nectapure | Buddleja Davidii Extract | | | 0.000 | |
| | | Thymus Vulgaris (Thyme) Flower/Leaf | | | 0.000 | |
| | | Extract | | | | |
| | | Water | | | 0.000 | Carrier |
| | | Glycerin | | | 0.000 | |
| | | Phenoxyethanol | | | 0.000 | Preservative |
| | | Methylparaben | | | 0.000 | Preservative |
| | | Ethylparaben | | | 0.000 | Preservative |
| | | Propylparaben | | | 0.000 | Preservative |
| | | Butylparaben | | | 0.000 | Preservative |
| | | Isobutylparaben | | | 0.000 | Preservative |
| | | Bis-Hydroxyethyl Tromethamine | | | 0.00 | |
| Active Organics | Actiphyte of Algae BG50P | Butylene Glycol | 0.200 | 39.50 | 0.079 | Moisturizer |
| | | Water | | 39.50 | 0.079 | Carrier |
| | | Phenoxyethanol | | 0.7260 | 0.001 | Preservative |
| | | Methylparaben | | 0.1550 | 0.000 | preservative |
| | | Ethylparaben | | 0.0390 | 0.000 | Preservative |
| | | Propylparaben | | 0.0200 | 0.000 | Preservative |
| | | Butylparaben | | 0.0400 | 0.000 | Preservative |
| | | Isobutylparaben | | 0.0200 | 0.000 | Preservative |
| | | Algae Extract | | 20.000 | 0.040 | Algae |
| Active Organics | Actiphyte of Boswellia | Butylene Glycol | 0.200 | 39.50 | 0.079 | Moisturizer |
| | BG50P | Water | | 39.50 | 0.079 | Carrier |
| | | Phenoxyethanol | | 0.7260 | 0.001 | Preservative |
| | | Methylparaben | | 0.1550 | 0.000 | Preservative |
| | | Ethylparaben | | 0.0390 | 0.000 | Preservative |
| | | Propylparaben | | 0.0200 | 0.000 | Preservative |
| | | Butylparaben | | 0.0400 | 0.000 | Preservative |
| | | Isobutylparaben | | 0.0200 | 0.000 | Preservative |
| | | Boswellia Serrata Resin Extract | | 20.000 | 0.040 | Boswellia |

| | | |
|---|---|---|
| Total | 100.000 | 99.900 |

The substrate 12 can be applied with the skin care composition by any suitable method known in the art, such as spraying, dipping, saturating, impregnating, brush coating, extruding, and the like. The amount of the skin care composition that may be added to the substrate may vary depending upon the type of substrate material utilized and the ingredients of the skin care composition. Generally, the substrate contains from about 150% to about 800% by weight of the skin care composition or greater.

In order to protect the skin care composition during packaging and to facilitate handling of the skin care delivery device, the device further includes the backing 14 as shown in FIG. 1. The backing generally comprises any suitable liquid impermeable material that is capable of releasably engaging the substrate 12. For instance, the backing 14 can permit handling of the substrate 12 which may be wet and slippery and that can otherwise be easily distorted during folding and other manipulation. The backing 14 also allows a user to handle the skin care delivery device without substantially contacting the skin care composition with one's hands. Not only does it prevent the composition from transferring to the hands of a user, but can also minimize contamination that can occur during such contact.

The backing 14, in one embodiment, can be made from a relatively thin polymer film. The polymer film, for instance, may comprise a single layer or may comprise multiple layers. In this regard, the polymer film can be monoextruded or coextruded.

Polymers that may be used to form the backing 14 include, for instance, polyolefins, such has polyethylene and polypropylene. Other polymers include a polyvinyl, a polyester, or mixtures thereof. It should also be understood that the above identified polymers also include any co-polymers of the materials.

Although optional, in one embodiment, the backing 14 can comprise a breathable film. For instance, the breathable film can comprise a microporous or monolithic film.

In one embodiment, the backing 14 can also include a release coating that faces and contacts the substrate 12. A release coating may be applied to the backing 14 in order to facilitate release of the backing from the substrate 12 during use. In one embodiment, for instance, the release coating may comprise a polymer wax or a silicone.

Another advantage to the backing 14 is that the backing allows the skin care delivery device 10 to be easily folded and unfolded. The skin care delivery device 10, for instance, may be folded prior to packaging so as to facilitate shipping. For instance, one embodiment of a process for folding the skin care delivery device 10 is illustrated in FIGS. 2, 3 and 4.

As shown in FIG. 2, for instance, in one embodiment, the substrate 12 can first be folded upon itself. In this manner, the backing 14 forms the outside layer of the folded product.

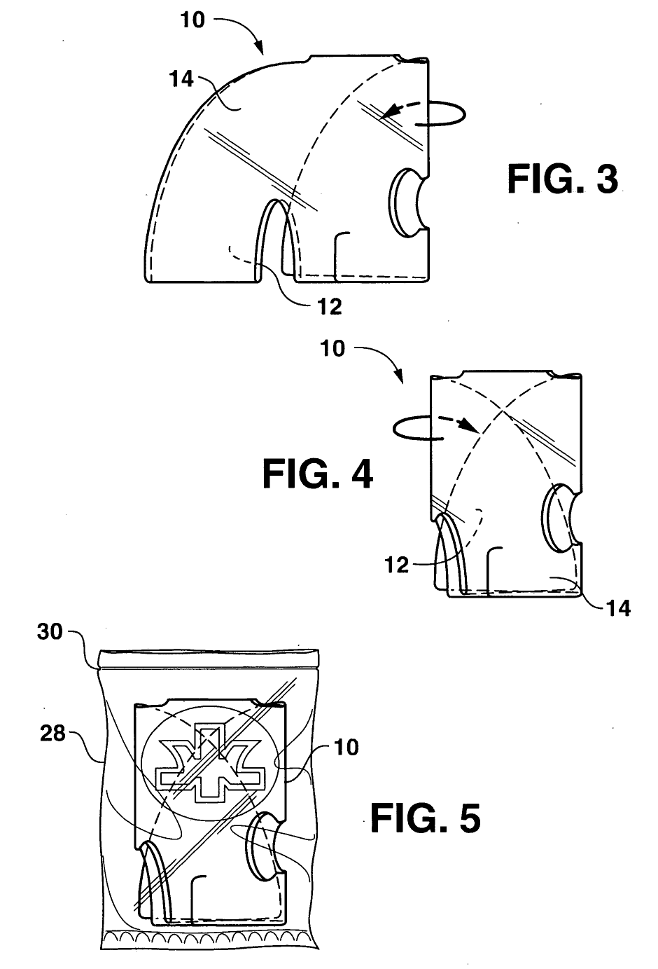

After the skin care delivery device 10 is folded in half as shown in FIG. 2, in one embodiment, the device can undergo a tri-fold. For instance, as shown in FIG. 3, the first fold of the tri-fold is completed. Referring to FIG. 4, the second fold of the tri-fold is shown producing a compact product for packaging. It should be understood, however, that any suitable folding configuration can be completed. For instance, more or less folds may be imparted on the device as desired.

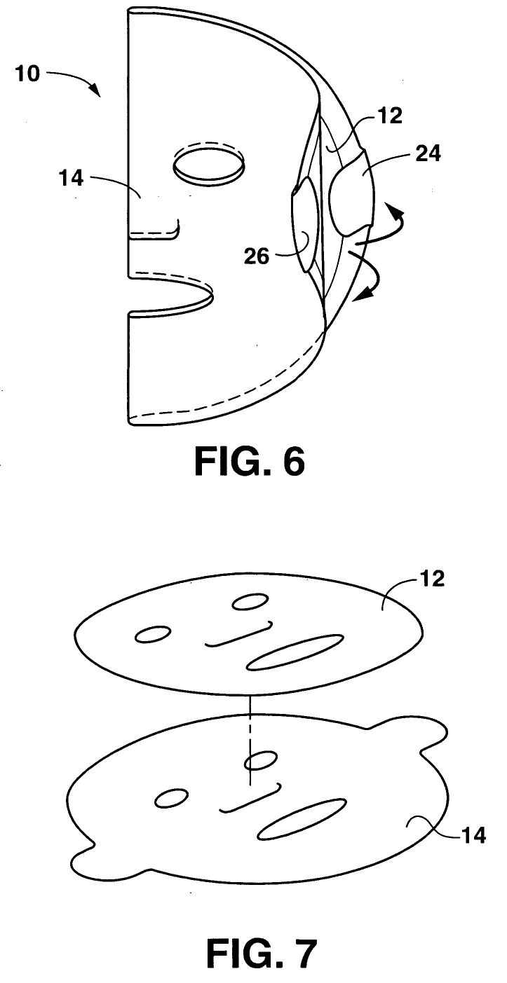

In one particular embodiment, the backing 14 can include one or more tab portions. For instance, as shown in FIG. 1, the backing 14 includes a first tab portion 24 and a second tab portion 26. In this embodiment, the tab portions 24 and 26 are on opposite sides of the skin care delivery device 10. The tab portions 24 and 26 can be folded over onto the substrate 12 prior to folding the substrate onto itself as shown in FIG. 2. In this manner, as shown in FIG. 6, the tab portion 24 becomes positioned in between the two faces of the substrate 12. The tab portion 24 thus provides a portion on the skin care delivery device that can be grasped by a user in order to unfold the device. More specifically, the tab portion 24 allows a user to grasp and pull apart the substrate 12 without having to contact the skin care composition present within the substrate. The tab portions also provide an area where the substrate has not otherwise attached to itself.

Once the skin care delivery device 10 is folded as desired as shown in FIG. 4, the skin care delivery device can be packaged as shown in FIG. 5. For instance, in one embodiment, the skin care delivery device 10 can be placed in a sealed package 28. The sealed package 28 can be made from any suitable material that is substantially liquid and gas impermeable. The purpose of the sealed package 28 is to prevent exposure of the skin care delivery device 10 to ambient air which may cause the product to dry out or to allow any of the skin care composition to leak from the container. The sealed package 28, for instance, can be made from polymer films and from polymer film laminates. In one embodiment, for instance, the sealed package 28 is made from a film laminate containing a metalized or foil layer.

If desired, the sealed package 28 can include a weakened portion 30 that allows one to easily open the package through a tearing motion. Alternatively, the sealed package may include a device that allows the package to be opened and closed in a sealed manner. For instance, in an alternative embodiment, the sealed package can include a groove and channel sealing member that extends across one side of the package.

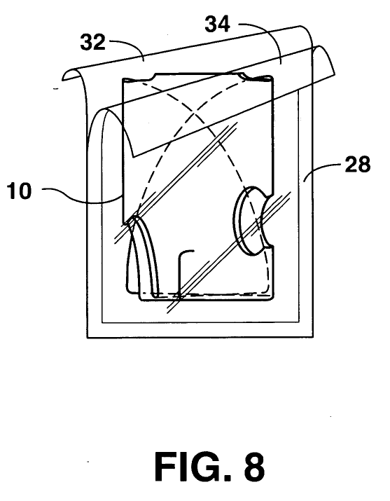

Referring to FIG. 8, still another embodiment of the sealed package 28 is shown. In this embodiment, the sealed package 28 includes a first flap 32 and a second flap 34 positioned along one end of the package. The flaps 32 and 34 are for grasping with a user's hand in order to pull apart the package and open the container for releasing the skin care delivery device 10.

The sealed package 28 can be translucent or can be one or more different colors. Various designs can also be printed on the package as desired. For instance, in one embodiment, logos and/or brand names may appear on the sealed package 28.

It should also be understood that the skin care delivery device 10 can be packaged with other various products as desired. The other products can be contained within the sealed package 28 in conjunction with the skin care delivery device 10 or can be otherwise sold as part of a kit that includes the sealed package. For instance, the skin care delivery device 10 can be packaged and sold with a complimentary product such as a second health and hygiene product that is different from the skin care delivery device. The other product can be, for instance, a cleaning formulation, a buff, or other such product.

In order to use the skin care delivery device 10 as shown in the Figures, in one embodiment, the device can first be removed from the sealed package. Once removed, the skin care delivery device 10 can be unfolded by manipulating the device using the backing 14. In one embodiment, for instance, the tab portions 24 and 26 can be used in order to unfold the skin care delivery device 10 and place the device against a user's skin.

Once properly positioned on a user's skin, the tab portions 24 and 26 can be pulled off the front surface of the substrate 12 and used to peel the backing 14 away from the substrate. Thus, the backing not only facilitates folding and unfolding of the device, but can also be used to properly position the device.

These and other modifications and variations to the present disclosure may be practiced by those of ordinary skill in the art, without departing from the spirit and scope of the present disclosure, which is more particularly set forth in the appended claims. In addition, it should be understood that aspects of the various embodiments may be interchanged either in whole or in part. Furthermore, those of ordinary skill in the art will appreciate that the foregoing description is by way of example only, and is not intended to limit the disclosure so further described in such appended claims.

What is claimed:

1. A disposable skin care delivery device comprising:
a substrate comprising a first surface and a second and opposite surface;
a skin care composition contained in the substrate and present at least at the first surface; and
a backing releasably engaged to the second surface of the substrate, and wherein the backing is configured to be removed from the substrate during application of the substrate to a user's skin, the backing including at least one tab portion to facilitate manipulation of the substrate.

2. A disposable skin care delivery device as defined in claim 1, wherein the backing comprises a polymer film.

3. A disposable skin care delivery device as defined in claim 1, wherein the backing comprises a monoextruded polymer film.

4. A disposable skin care delivery device as defined in claim 2, wherein the polymer film comprises a polyolefin, a polyester, a polyvinyl, mixtures thereof, or laminates thereof.

5. A disposable skin care delivery device as defined in claim 1, wherein the substrate comprises a nonwoven web.

6. A disposable skin care delivery device as defined in claim 5, wherein the nonwoven web contains a mixture of pulp fibers and synthetic fibers.

7. A disposable skin care delivery device as defined in claim 6, wherein the nonwoven web comprises a coform web.

8. A disposable skin care delivery device as defined in claim 5, wherein the nonwoven web comprises a hydroentangled web, a bonded carded web, an air-formed web, a meltblown web, or a spunbond web.

9. A disposable skin care delivery device as defined in claim 1, wherein the substrate has a shape configured to cover a portion of a person's face.

10. A disposable skin care delivery device as defined in claim 9, wherein the substrate includes a nose portion and an opening configured to surround a person's mouth.

11. A disposable skin care delivery device as defined in claim 10, wherein the backing also includes a corresponding mouth opening.

12. A disposable skin care delivery device as defined in claim 1, wherein the backing includes a first tab portion and a second tab portion, each of the tab portions being folded over onto the first surface of the substrate.

13. A disposable skin care delivery device as defined in claim 1, wherein the skin care composition comprises at least a humectant and a moisturizer.

14. A disposable skin care delivery device as defined in claim 1, wherein at least a portion of the substrate is folded onto itself.

15. A disposable skin care delivery product comprising:
a sealed package; and
a disposable skin care delivery device comprising a substrate having a first surface and a second and opposite surface, the substrate containing a skin care composition that is present at least at the first surface, the second surface being releasably engaged by a backing and wherein at least a portion of the substrate is folded on itself within the sealed package, the backing including at least one tab portion, the tab portion being folded over onto the first surface of the substrate, the tab portion being configured to be held by a user for unfolding the disposable skin care delivery device prior to use.

16. A disposable skin care delivery product as defined in claim 15, wherein the substrate comprises a nonwoven web and the backing comprises a polymer film.

17. A disposable skin care delivery product as defined in claim 16, wherein the nonwoven web contains a mixture of pulp fibers and synthetic fibers.

18. A disposable skin care delivery product as defined in claim 16, wherein the skin care delivery device has a shape configured to cover a portion of a person's face, the skin care delivery device defining an opening in the substrate and the backing that is configured to surround a mouth of a user.

19. A disposable skin care delivery product as defined in claim 16, wherein the backing includes a first tab portion and a second tab portion, each of the tab portions being folded over onto the first surface of the substrate, the tab portions being positioned generally at opposite sides of the skin care delivery device.

20. A disposable skin care delivery product as defined in claim 15, wherein the sealed package is made from at least one polymer film.

21. A method of treating the skin of a user comprising:
removing from a sealed package a disposable skin care delivery device, the skin care delivery device comprising a substrate having a first surface and a second and opposite surface, the substrate containing a skin care composition that is present at least at the first surface, the second surface of the substrate being releasably engaged by a backing, the backing including at least one tab portion that is folded over onto the first surface of the substrate, the substrate comprising a nonwoven web and the backing comprising a polymer film, the disposable skin care delivery device being present in the sealed package in a folded condition;
unfolding the disposable skin care delivery device and placing the first surface of the substrate against a user's skin; and
removing the backing from the second surface of the substrate.

22. A method as defined in claim 21, wherein the skin care delivery device has a shape configured to cover a portion of a person's face, the skin care delivery device defining an opening configured to surround the mouth of a user, the opening extending through the substrate and the backing.

23. A method as defined in claim 22, wherein the backing includes a first tab portion and a second tab portion, each of the tab portions being folded over onto the first surface, the first surface being folded onto itself within the sealed package and wherein the disposable skin care delivery device is unfolded by grasping the tab portions.
